(19) United States
(12) Patent Application Publication
(10) Pub. No.: US 2002/0009755 A1
(43) Pub. Date: Jan. 24, 2002

(54) ALTERNATIVE SPLICING OF FIBROBLAST GROWTH FACTOR RECEPTOR 2 MRNA IN PROSTATE CANCER

(76) Inventors: Mariano A. Garcia-Blanco, Durham, NC (US); Russ P Carstens, Durham, NC (US)

Correspondence Address: NIXON & VANDERHYDE PC, ATTORNEY AT LAW, 1100 NORTH GLEBE ROAD 8TH FLOOR, ARLINGTON, VA, 222014714

(21) Appl. No.: 09/465,802

(22) Filed: Dec. 17, 1999

Related U.S. Application Data
(60) Provisional application No. 60/112,856, filed on Dec. 17, 1998.

(51) Int. Cl. G01N 33/53; G01N 33/567; G01N 33/574; C07H 21/02; C07H 21/04; C12P 19/34; C07K 16/00

(52) U.S. Cl. 435/7.2; 435/91.2; 536/23.1; 536/24.33; 530/387.1

(57) ABSTRACT

The present invention relates, in general, to prostatic cancer and, in particular, to a method of monitoring progression of prostate cancer and to a method of treating the disease.

This application claims the benefit of U.S. Provisional Application No. 60/112,856, filed Dec. 17, 1998, the entire content of which is hereby incorporated by reference in this application.

This invention was made with Government support under Grant No. GM49639 and ES69773 awarded by the National Institutes of Health; 96002290 awarded by the American Heart Association; DK07731 awarded by the (NIDDK) National Institutes of Diabetics and Digestive and Kidney Diseases. The Government has certain rights in the invention.

### TECHNICAL FIELD

The present invention relates, in general, to prostatic cancer and, in particular, to a method of monitoring progression of prostate cancer. The invention further relates to a method of identifying factors responsible for the progression of an androgen sensitive prostatic tumor to an androgen insensitive tumor. The invention also relates to a method of screening compounds for their ability to inhibit prostate cancer progression.

### BACKGROUND

Prostate cancer, the second most common cause of male cancer deaths, remains a therapeutic dilemma. While there is a high prevalence of the disease, based on autopsy studies, a substantial percentage of such cancers will never threaten a patient's well-being, prompting some to recommend expectant management (rather than aggressive definitive therapy) as the best treatment option for many elderly males. In others, prostate cancer progresses with early metastatic spread, confounding curative intervention of localized disease. Elucidation of the biological processes that cause prostate cancer to both metastasize and develop androgen independent growth is needed. Recognition of an aggressive phenotype using biological markers might allow the clinician to limit aggressive therapy for those at risk, while sparing those with indolent disease. In addition, better understanding of the mechanisms that lead to prostate cancer progression can guide the development and testing of new prostate cancer therapies.

An important area of research that has had a significant impact upon the understanding of carcinogenesis, both in the prostate as well as other tissues, has been identification of growth factors involved in growth of normal and cancerous cells (Aaronson, Science 254:1146-1153 (1991)). In the prostate, such polypeptide growth factors play a critical role in mediating the interactions between stromal and epithelial cells which have been shown to be important in maintaining normal prostate growth and differentiation (Cunha et al., Cell Diff. 17:137-148 (1985); Cunha et al., Endocrine Rev. 8:338-363 (1987); Gleave et al., J. Urol. 147:1151-1159 (1992); Yan et al., Mol. Endo. 6:2123-2128 (1992)). It is proposed that growth factors are key elements that mediate the effects of androgens on prostate cell growth and differentiation via a directional communication system from stromal to epithelial cells (Yan et al., Mol. Endo. 6:2123-2128 (1992). Thus, disruption of this system of cellular communication through interactions of growth factors with their receptors may be a step in tumor development and/or progression.

One of the families of growth factors that have been implicated in normal and cancerous cell growth in the prostate is the fibroblast growth factor (FGF) family. These mitogens consist of over ten factors, FGF-1 through FGF-9 (Mason, Cell 78:547-552 (1994)). Several of these factors have recently been shown to play an important role in prostate cell growth (Mansson et al., Cancer Res. 49:2485-2494 (1989); McKeehan, 1991). In particular, fibroblast growth factor 7 (FGF7, or keratinocyte growth factor(KGF)) has been shown to be a potential mediator of the effects of androgen on prostate growth (Yan et al., Mol. Endo. 6:2123-2128 (1992). FGF-7 expression is limited to stromal cells in a variety of tissues and it is mitogenic for epithelial cells (Rubin et al., Proc. Natl. Acad. Sci. USA. 86:802-806 (1989); Finch et al., Science 245:752-755 (1989)). In the prostate, production of FGF-7 is similarly restricted to stromal cells and is induced by androgens (Yan et al., Mol. Endo. 6:2123-2128 (1992). Epithelial cells, but not stromal cells, express a specific receptor for FGF-7. This receptor is a specific isoform of fibroblast growth factor receptor 2 (FGF-R2), a member of the tyrosine kinase family of cell surface receptors (Hattori et al., Proc. Natl. Acad. Sci. USA 87:5983-5987 (1990); Miki et al., Science 251:72-75 (1991); Miki et al., Proc. Natl. Acad. Sci. USA 89:246-250 (1992); Johnson et al., Cellular Biology 11(9):4627-4634 (1991)). In normal prostate, and in prostate malignancies as well, it appears that the interaction between FGF-7 and this receptor plays a part in maintaining androgen responsive growth and differentiation of epithelial cells (Yan et al., Mol. Endo. 6:2123-2128 (1992); Yan et al., Mol. Cell Biol. 13:4513-4522 (1993)).

There are currently four known fibroblast growth factor receptor genes, FGF-R1-4, which share significant structural and amino acid homology (Miki et al., Proc. Natl. Acad. Sci. USA 89:246-250 (1992); Johnson et al., Cellular Biology 11(9):4627-4634 (1991); Dionne et al., The EMBO Journal, 9(9):2685-2692 (1990); Jaye et al., Bioch. Biophys. Acta 1135:185-199 (1992); Johnson and Williams, Adv. Cancer Res., 60:1-41 (1993)). The general structure of these receptors consist of two or three Ig-like domains which appear to be involved in ligand binding, a single transmembrane domain, and intracellular tyrosine kinase domains. Although a number of different isoforms of each of these receptors have been demonstrated, the best characterized example of post-transcriptional modification of these receptors is that involving the second half of the third Ig-like domain of FGF-R2. Here there is a mutually exclusive splicing event in which the second half of this domain is comprised of either the 148 nucleotide IIIb exon or the 145 nucleotide IIIc exon. The resulting isoforms differ in their ligand specificity in a functionally important manner. The IIIb containing form of the receptor (also known as keratinocyte growth factor receptor, or KGF-R) is responsive to FGF-7 whereas FGF-R2 IIIc is not. Conversely FGF-R2 IIIc displays a much greater affinity for FGF-2 (basic FGF) than FGF R2 IIIb does (Miki et al., Proc. Natl. Acad. Sci. USA 89:246-250 (1992); Bottaro et al., J. Biol. Chem. 265:12767-12770 (1990); Yayon et al., EMBO J. 11:1885 1890 (1992)). Not surprisingly, these differences in ligand specificity are consistent with the cell type distribution seen for these receptor isoforms. The IIIb isoform is the predominant isoform seen in epithelial cell types, including prostatic epithelia, and the IIIc isoform is expressed in some, but not all, cells of mesenchymal origin (Miki et al., Proc. Natl. Acad. Sci. USA 89:246250 (1992); Yan et al., Mol. Cell Biol. 13:4513-4522 (1993); Orr-Urtreger et al., Dev. Biol. 158:475-486 (1993)). Thus, this alternative splicing process is consistent with a directional pathway of androgen stimulation in the prostate, whereby androgen mediated stimulated secretion of FGF-7 results in proliferation and differentiation of prostatic epithelial cells; effects which are effected through the IIIb isoform of the FGF-R2 receptor.

McKeehan and colleagues have used the transplantable Dunning R3327 rat prostate tumor model to gain further insight into the mechanisms involved in the development of androgen insensitivity (Yan et al., Mol. Endo. 6:2123-2128 (1992); Yan et al., Mol. Cell Biol. 13:4513-4522 (1993); Isaacs et al., Cancer Res. 38:4353-4359 (1978)). The androgen sensitive, well differentiated R3327PAP, or DT, tumor can progress to an androgen insensitive, highly aggressive R3327AT3 tumor after castration and serial passage through castrated and female hosts. A very interesting feature of these tumors is that the switch from an androgen sensitive, slow growing tumor to a more aggressive, androgen insensitive tumor is accompanied by a change in the alternative splicing pattern of the epithelial cell FGF-R2 receptor (Yan et al., Mol. Cell Biol. 13:4513-4522 (1993)). Normal prostate epithelia, as well as DT tumor cells, express exclusively the FGF-R2 IIIb isoform as described above. In contrast, AT3 tumors express the FGF-R2 IIIc variant which, as discussed previously, is not responsive to stromal derived FGF-7. Thus, loss of responsiveness to FGF-7 may render these cells independent of stromal derived factors and set up autocrine loops between FGF ligands and their receptors allowing autonomous growth and progression to a more malignant phenotype (Yan et al., Mol. Cell Biol. 13:4513-4522 (1993)).

### SUMMARY OF THE INVENTION

The present invention relates to a method of monitoring progression of prostate cancer. The invention further relates to a method of identifying factors responsible for the progression of an androgen sensitive prostatic tumor to an androgen insensitive tumor.

### BRIEF DESCRIPTION OF THE DRAWINGS

FIGS. 1A and 1B. Structural organization of the FGF-R2 gene and demonstration of IIIb and IIIc mutually exclusive splicing. (FIG. 1A) Organization of the FGF-R2 protein domains (top) and genomic gene arrangement of the region in which alternative splicing yields either IIIb or IIIc exon containing transcripts encoding the second half of the third Ig-like domain. TM=transmembrane domain. TK=tyrosine kinase domains. The solid box indicates a highly acidic domain and the thick line indicates the IIIb- or IIIc-encoded portion of the protein. Shaded boxes indicate exons and solid lines represent introns with intron sizes indicated (bottom). U and D indicate the exons upstream and downstream of these alternative exons, respectively. (FIG. 1B) Scale representation of the exons (solid boxes) and introns (solid lines) with regions of high (at least 90%) rat: human intron sequence similarity (shaded boxes). Also shown are regions FS and FL and their associated sizes.

FIGS. 2A and 2B. Splicing of the endogenous gene transcript in DT3 and AT3 cells. (FIG. 1A) Map illustrating PCR products containing exon IIIb or IIIc amplified with primers FGF-FB and FGF-RB and sizes (in nucleotides) of fragments which result from Ava I or Hinc II digestion. U, upstream exon; D, downstream exon. (FIG. 1B) Gel showing the RT-PCR products following digestion with Ava I and Hinc II. DT3 cells express only IIIb containing products and AT3 cells express IIIc containing products. U=Uncut products. A=Ava I digested products. H=Hinc II digested products. M=pBR322/Msp I DNA size markers.

FIGS. 3A-3E. Rat FGF-R2 minigenes transfected into DT3 and AT3 cells reproduce the splicing pattern of the endogenous gene. (FIG. 3A) Representation of the two exon, one intron splicing construct pI-11 and insertion of FGF-R2 genomic sequences FL and FS (which were generated using the primer sets indicated at bottom) to create minigenes pI-11-FL and pI-11-FS, respectively. CMV indicates the efficient immediate early CMV promoter and pA the bovine growth hormone polyadenylation sequence. The Xba I and Xho I sites used for cloning and the T7 and SP6 vector specific primers are also indicated. U indicates the 5′ exon of pI-11 and D the 3′ exon of pI-11. (FIG. 3B) pI-11 prem-RNA is spliced almost completely and with equal efficiency in DT3 and AT3 cells, indicating no differences in the ability of these cells to splice the exons. RT-PCR products for this and subsequent minigenes were obtained using the T7 and SP6 promoter primers. (FIGS. 3C and 3D) Minigenes pI-11-FL and pI-11-FS reproduce the endogenous gene splicing pattern. The major PCR product containing either IIIb (3B or B) or IIIc (3C or C) is 380 or 377 bp, respectively. Products containing exons U, IIIb , IIIc , and D are indicated to the right. The sizes of products of Ava I and Hinc II digestion are also indicated. Quantification was performed to yield values for the fraction of the expected IIIb (in DT3) or IIIc (in AT3) exon as a fraction of IIIb and IIIc containing products and also as a fraction of products skipping IIIb and IIIc . (FIG. 3E) Representation of the origin of the products obtained when UBD, UCD, and UBCD products are cut with Ava I and Hinc II. Sizes are indicated in base pairs. Lanes are labeled as in FIG. 2.

FIGS. 4A-4C. Deletions which result in loss of sequences between the Nde I and Nsi I sites in intron 2 result in loss of regulation in DT3 cells. (FIG. 4A) The IIIb and IIIc exons (solid boxes) and the intron (intron 2) between them (solid line) are shown. Also indicated are the restriction enzymes used to generate these deletions and the regions of high rat: human sequence homology (shaded boxes). The locations of these restriction sites are represented as the position (in nucleotides) from the start of the intron and are measured to the center position of each recognition sequence. The minigenes tested consisted of deletions (hatched boxes) from parent construct pI-11-FS and results of these deletions in DT3 cells are summarized. Delta, construct in which the sequence between the indicated restriction enzymes was deleted from pI-11-FS; plus, deletion constructs which still demonstrated >80% IIIb inclusion in DT3 cells; minus, deletion constructs with ≦55% IIIb inclusion in DT3 cells. (FIG. 4B) Results of the most representative intron 2 deletions in DT3 cells. Deletion of over half of the intron from Bcl I to Nde I did not affect regulation, whereas deletions spanning Nde I to Nsi I caused loss of regulation. A deletion of Nsi I to Stu I sequences also caused some loss of regulation, but less than a Nde I to Nsi I deletion. (FIG. 4C) The same deletions had no effect upon splicing in AT3 cells. Efficient IIIc usage was seen in these deletions, as well as all deletions summarized in FIG. 4A. Abbreviations are defined in the descriptions of FIG. 2 and FIG. 3.

FIGS. 5A and 5B. Sequences contained between the Nde I and Nsi I sites of intron 2 normally function to activate upstream IIIb splicing and repress downstream IIIc splicing. (FIG. 5A) Method used to generate minigene constructs containing either the IIIb or IIIc exon with Nde I to Nsi I sequences (crosshatched boxes) present or deleted. All constructs were deleted of sequences Bcl I to Nde I which were previously shown to be dispensable for regulation. Primers used to generate these regions in relation to the sequences of pI-11-FS are shown. (FIG. 5B) Transfection of these minigenes into DT3 and AT3 cells reveals that AT3 cells use exon IIIc highly efficiently and do not use exon IIIb efficiently regardless of the presence of Nde I to Nsi I sequences. DT3 cells use exon IIIb efficiently only when these Nde I to Nsi I sequences are present downstream. DT3 cells do not use exon IIIc efficiently, but when these sequences are deleted, IIIc usage triples.

FIGS. 6A-6D. A critical 18 nuclotide sequence within the 57 nucleotide ISAR sequence between Nde I and Nsi I nearly restores splicing regulation in DT3 cells. (FIG. 6A) The 57 nucleotide ISAR sequence is indicated at the top and deletions and mutants of this sequence are shown as are control pBluescript sequences. The 18 nucleotide “core sequence” (Rep1) is boxed, and mutant sequences are underlined and in boldface. All sequences were tested by deleting ISAR sequences from pI-11-FS/Not/Cla-ISAR and inserting the indicated sequences. (FIG. 6B) SAR-20 and SAR 3′ sequences restore regulation, whereas SAR 5′ does not. (FIG. 6C) Mutations in the 18 nucleotide sequence shared by SAR-20 and SAR 3′ (Mut 2 and Mut 3) cause loss of regulation whereas a mutation outside this region (Mut 1) preserves regulation. (FIG. 6D) One or three copies of the 18 nucleotide “core sequence” restore splicing regulation, with three repeats of the sequence being slightly more efficient than one repeat. Abbreviations are defined in descriptions of FIGS. 2 and 3.

FIG. 7. DT3 cells contain a titratable factor or factors required for appropriate splicing regulation which can be overcome in transient transfections. Transient transfection of DT3 cells with increasing amounts of pI-11-FS minigenes resulted in stepwise loss of IIIb inclusion and increased IIIc inclusion, suggesting that a factor or factors required for regulation (i.e., IIIb inclusion and/or IIIc exclusion) is overwhelmed when large amounts of these minigenes are transfected.

FIGS. 8A and 8B. Intron sequences important for regulation of rat and human FGF-R2 splicing display are highly similar. (FIG. 8A) Rat intron sequences corresponding to previously reported 21 nucleotide human sequence, IAS 2, which also mediates IIIb activation contain only one nucleotide difference. (FIG. 8B) The 57 nucleotide rat ISAR sequence is highly similar to human sequences in this same region, including the 18 nt shown to be most important for regulation (boxed sequences).

FIG. 9. Depiction of a model which can account for results and the high fidelity of FGF-R2 splicing. AT3 cells use a default splicing pathway and choose the IIIc exon because of its stronger poypyrimidine tract (ppt), they splice IIIb inefficiently due to its weaker polypyrimidine tract. DT3 cells require regulatory factor(s) which can activate (+) the weaker IIIb exon and at the same time repress use of the IIIc exon. The ISAR element (indicated by the hatched box) is shown binding a factor or complex of factors (large shaded oval) which mediates both of these effects. The previously demonstrated contributions of other cis-elements and associated factors (smaller shaded ovals) on IIIb activation are also shown, as well as the suggestion of possible cooperative interaction between proteins bound at several locations within the intron. Abbreviations are defined in the description to FIG. 3.

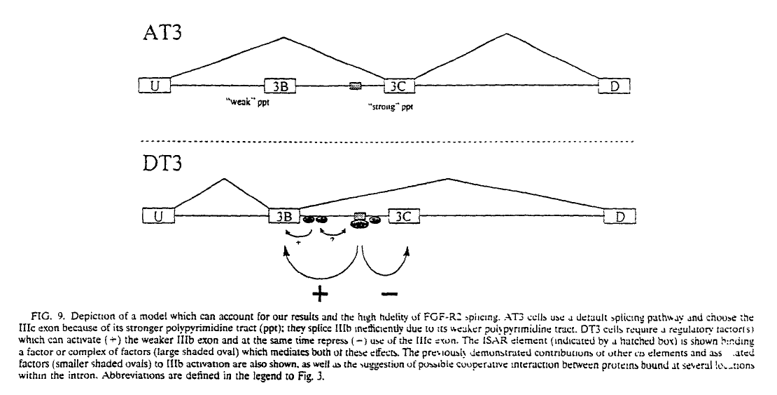

FIG. 10. Rat FGF-R2 intron between exons IIIb and IIIc.

FIG. 11. Human FGF-R2 intron between exons IIIb and IIIc.

### DETAILED DESCRIPTION OF THE INVENTION

Progression of human prostate cancer from an androgen sensitive to an androgen insensitive tumor is accompanied by a change in alterative splicing of FGF-R2. This change results in a loss of the FGF-R2 IIIb isoform and predominant expression of the FGF-R2 IIIc isoform. This event provides an important biological marker for progression of prostate cancer.

The present invention relates to a method of determining the metastatic potential of a prostate tumor in a patient. The invention also relates to a method of assessing the androgen sensitivity of such a tumor. Both methods comprise assaying for the expression in the tumor of the IIIc isoform of FGF-R2. The analysis can be carried out using a variety of techniques. As will be clear from the Examples that follow, the presence of the FGF-R2 IIIc transcript can be readily detected using an RT-PCR based assay. Consistent with this assay, total cellular RNA from the tumor can be isolated using standard procedures and that RNA can then be reverse transcribed using, for example, a commercial kit. An aliquot of the reverse transcription reaction can then be amplified using, for example, standard PCR techniques. Appropriate primers for use in the amplification include those referenced in the Examples, and comparable primers designed based on corresponding human sequences. The PCR product can then be subjected to restriction endonuclease digestion using restriction endonucleases that distinguish between the IIIb and IIIc isoforms of FGF-R2. The Examples that follow describe the use of Aval and HincII, there being a single AvaI site in exon IIIb but none in exon IIIc , whereas IIIc contains 2 HincII sites not present in IIIb . Thus, PCR products containing IIIb yield full length products that are not digested by HincII and yield 2 fragments upon digestion with AvaI. IIIc containing PCR products result in a full length product that is not cut by AvaI and 3 fragments upon digestion within HincII. Standard polyacrylamide gel electrophoresis can be used, for example, to analyze the restriction endonuclease products (see Examples).

In addition to the foregoing technique, expression of IIIb and IIIc can be detected using sequences complementary to the IIIb and IIIc encoding sequences, e.g., attached to a solid support (e.g., a chip). In accordance with this approach tissue or cell extracts, for example, can be contacted with the support under conditions such that hybridization of IIIb or IIIc encoding sequences in the extract to the support bound complementary sequence can occur, and then the presence or absence of a hybridization product can be detected using any of a variety of standard techniques.

While the above-described techniques are nucleic acid based, it will be appreciated that antibodies (monoclonal or polyclonal) specific for the respective isoforms can be used in situ or in extracts. Immunohistochemical assays can be readily developed based on the antibodies produced, for example, in accordance with the method described in Example 2. Multiple tissue fixation, washing and developments techniques can be tested for in situ detection and optimum conditions selected. The conversion of Western data to histochemistry can be expected to follow standard algorithms.

The tumor sample used in accordance with the above method can be fresh or frozen. Alternatively, cells derived from the tumor can be expanded in culture or in a host animal, such as a nude mouse, prior to use in the above methods.

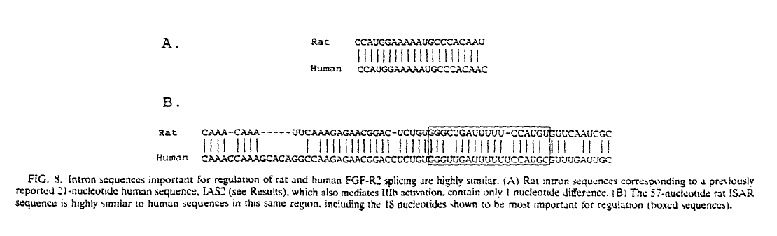

The present invention also relates to a method of identifying factors (e.g., splice regulators) responsible for the progression of prostatic tumors to an aggressive (metastatic) phenotype. In accordance with this method, an ISAR sequence (see FIG. 8B), preferably a human ISAR sequence, or portion thereof responsible for FGF-R2 splice regulation (e.g., a portion that includes the 18 nucleotide sequence boxed in FIG. 8B), can be contacted with a source of such factors. Sources include, for example, a whole or fractionated extract (e.g., a nuclear extract) of IIIb producing prostatic epithelial cells (for example, a DT3 cell nuclear extract). Binding of a factor in the factor source to the ISAR sequence, or portion thereof, can be detected using standard protocols including UV crosslinking (Garcia-Blanco et al, Genes Devel. 3:1874 (1989)), RNA mobility shift assays (Jamison et al, Proc. Natl. Acad. Sci. 89:5482 (1992)); and RNA affinity columns (Garcia-Blanco et al, Genes Devel. 3:1874 (1989)). Factors that bind the ISAR sequence, or portions thereof, are potential FGF-R2 splicing regulators.

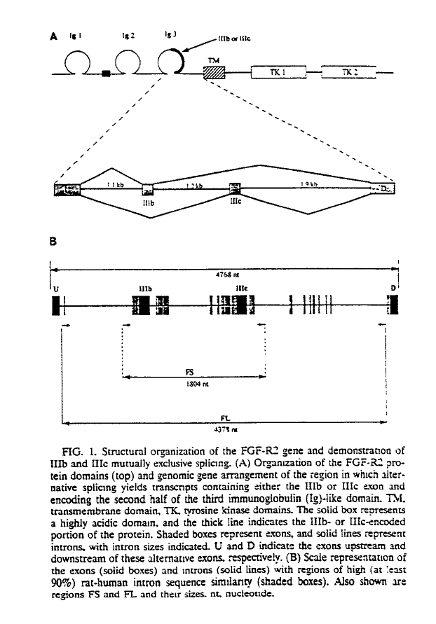

Alternatively, an in vitro splicing system that recapitulates the regulation of alternative slicing observed for the FGF-R2 transcripts can be used. In accordance with this approach, a splicing substrate is used comprising, for example, the upstream and downstream exons shown in FIG. 1, sequences surrounding and including the IIIb and IIIc encoding sequences and the intervening intron. The splicing substrate can be incubated with a source of FGF-R2 splice sequence regulators. Examples of appropriate sources are described above. An RT-PCR assay, for example, can be used to detect the results of the in vi tro splicing.

As indicated above, fractions of cell extracts can be used as sources of splice regulators. Such fractions can be prepared, for example, using a series of conventional chromatographic matrices (e.g., ion exchangers). Active chromatographic fractions, or whole extracts, can be applied to RNA affinity matrices (e.g., RNA's containing ISAR sequences or portions thereof as described above) to effect further purification of the factor(s) responsible for splice regulation (see, for example, Bohjanen et al, Nucl. Acids Res. 24:3733 (1996)). Splice regulators identified in accordance with these methods are encompassed within the scope of the invention.

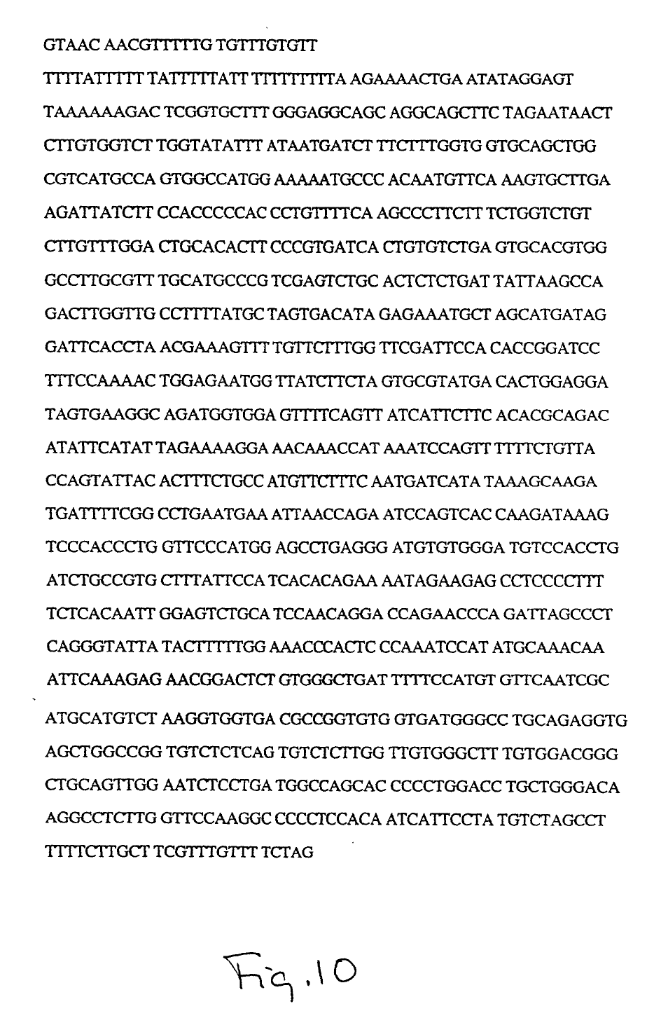

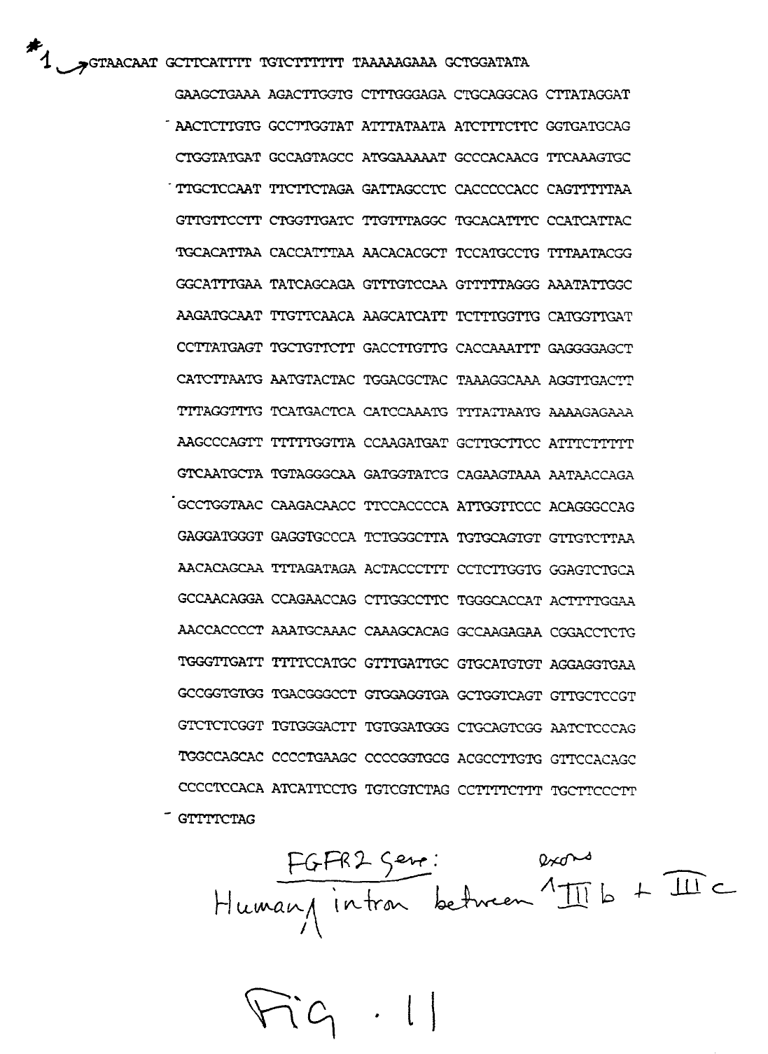

The invention also relates to nucleic acid sequences, RNA and DNA, used in the above-described methods, including the ISAR sequences shown in FIG. 8 and portions thereof, particularly splice regulatory portions thereof (e.g., portions that include the 18 nucleotide sequence boxed in FIG. 8) and the IIIb and IIIc encoding sequences (see also sequences of FIG. 10 and FIG. 11). The nucleic acids can be present in pure form or incorporated into a vector. The nucleic acids can be bound to a solid support. The invention further includes kits suitable for use in the above methods, which kits include the above nucleic acids (particularly, the human sequences), for example, disposed within a container means.

While the present invention is described with reference to alternative splicing in prostate cancer, alternative splicing is likely to be involved in other types of neoplasms, including bladder cancer.

Certain aspects of the present invention are described in greater detail in the non-limiting Examples that follow. (See also Carstens et al, Oncogene 15:3059 (1997); Carstens et al Mol. Cell. Biol. 18:2205 (1998)—the entire contents of both of these references being incorporated herein by reference).

### EXAMPLE 1

### Identification of Intronic Sequence Element

Expermental

Plasmid Construction.

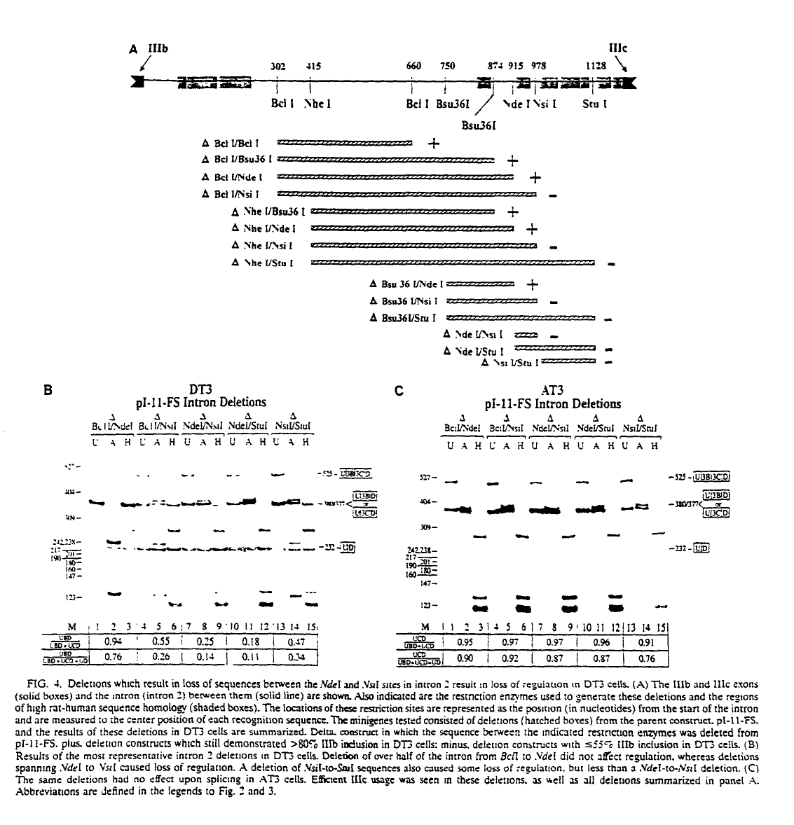

The pPIP11 adenoviral splicing construct is based on the L1 and L2 exons and is similar to the previously described pPIP7A (36), except that the sequence of the 3′exon between the Pst I sites and Hind III sites has been replaced by the sequence 5′-CTGCAGGACAAACTCTTCGCGGTCTCTATGCATCCTCCGAACGGTAAGACCC TAAGCTT-3′. The sequences of pPIP11 were PCR amplified with primers PIP10-F (5′-CCCGGGGGTACCGGGCGAATTCGAATT CGAGCTCACTC-3′) and PIP11-R (5′-CCCGGGACTAGTAAGCTTAGGCT CTTGGCGTT-3′). The PCR product was digested with EcoR1 and Spe I and inserted into the EcoR1 and Xba I sites of the eukaryotic expression vector PCDNA3 (Invitrogen). All cloning was done using standard methodologies. PCR amplification of genomic DNA from AT3 cells with FGF-R2 specific primers Int 3BF2 5′-CCGGACTAGTCACTACCGTTCTCCACCACT-3′and Int 3CR 5′-CCGGCTCGAGGGTCGGAAATCATTCGAAAC-3′, and Intron 1F-5′ CCGGACTAGTAAGCCCAAGGGGCCAGCAGT-3′and Intron 3R 5′ CCGGCTCGAGACGAAGAG CCAAGGGCGCCT-3′ yielded fragments FL and FS, respectively (FIG. 1B). These products were digested with Spe I and Xho I and inserted into the Xba I and Xho sites of the pI-11 intron to yield pI-11-FL and pI-11-FS. Constructs derived from intron deletions in pI-11-FS as represented in FIG. 4A (for example, pI-11-FS-ΔBcl-I/Nde I) were obtained by first cloning the FS sequences into the Spe I and Xho 1 sites of pBluescript (Stratagene), to generate pBlue-FS since these enzymes cut within PCDNA3 but not pBluescript. Deletions were performed by sequential digestion of pBlue-FS using the indicated restriction endonucleases. The digested ends were blunted using Pfu polymerase (Stratagene) and the resulting plasmids were gel purified, and religated using T4 DNA ligase. These plasmids were digested with Spe I and Xho I and the minigenes were cloned back into the Xba I and Xho sites of pI-11. Plasmids pI-11-IIIb -plus and pI-11-IIIb-minus were obtained using primers Int 3BF2 and Intron 2R2 5′-CCGGCTCGAGGGCTAGACATAGGAATGATT-3′ and PCR amplification of pI-11-FS-ΔBcl I/Nde I and pI-11-FS-ΔBcl I/Nsi I, respectively. After Spe I and Xho I digestion the PCR products were cloned into the Xba I and Xho I sites of pI-11. Plasmids pI-11-IIIc-plus and pI-11-IIIc-minus were similarly obtained by PCR amplification using primers Intron 2F 5′ C.CGGACTAGTCAACGTTTTTGTGTTTGTGT-3′ and Int 3CR to amplify pI-11-FS-ΔBcl I/Nde I and pI-11-FS-ΔBcl I/Nsi I, respectively, followed by digestion with Spe I and Xho I, and insertion into the Xba I and Xho I sites of pI-11. pI-11-FS-Not/Cla-ISAR resulted from PCR of pI-11-FS using primers Nde/Not-F 5′-CCGGCATATGGCGGCCGCC AAACAAATTCAAAGAGAAC-3′and Nsi/Cla-R 5′-CCGGATGCATATCGATGCGATTGAA CACATGGAAAA-3′, digestion of these products with Nde Iand Nsi I and cloning into the Nde I and Nsi I sites in pBlue-FS. The minigene sequence was removed with Spe I and Xho I and cloned into the Xba I and Xho I sites of pI-11 to generate pI-11-FS-Not/Cla-ISAR. The ISAR mutant constructs pI-11-Not/Cla: Bluel, SAR 5′, SAR 3′, SAR-20, Mut 1, Mut 2, Mut 3, Blue2, Rep1, and Rep 3 were obtained by deleting ISAR from pI-11-FS-Not/Cla-ISAR using Not I and Cla I, and then inserting annealed oligonucleotides with complementary Not I and Cla I sites as represented by the following oligonucleotide pairs: Bluel-F:

5′-GGAAGCGACTCCCCGTCGTGTAGATAACTACGATACGGGAGG GCTTACCATCTGGCCCCAGTGAT-3′; Bluel-R:

5′-CGATCACTGGGGCCAGATGGTAAGCCCTCCCGTATCGTAGTTA TCTACACGACGGGGAGTCGC-3′; SAR 5′-F:

5′-GGCCGCAAACAAATTCAAAGAGAACGGACTCTGTAT-3′; SAR 5′-R:

5′-CGATACAGAGTCCGTTCTCTTTGAATTTGTTTGGC-3′; SAR 3′-F:

5′-GGCCGCGGGCTGATTTTTCCATGTGTTCAATCGCAT-3′; SAR 3′-R:

5′-CGATGCGATTGAACACATGGAAAAATCAGCCCGC-3′; SAR-20-F:

5′-GGCCGCCAAAGAGAACGGACTCTGTGGGCTGATTTTTCCATGTAT-3′; SAR-20-R:

5′-CGATACATGGAAAAATCAGCCCACAGAGTCCGTTCTCTTTGGC-3′; Mutl-F:

5′-GGCCGCCAAACTCTACGGACTCTGTGGGCTGATTTTTCCATGTAT-3′; Mutl-R:

5′-CGATACATGGAAAAATCAGCCCACAGAGTCCGTAGAGTTTGGC-3′; Mut2-F:

5′-GGCCGCCAAAGAGAACGGACTCTGTGGGCTGAAAGATCCATGTAT 3′; Mut2-R: 5′-CGATACATGGATCTTTCAGCCCACAGAGTCCGTTCTCTTTGGC-3′; Mut3-F:

5′GGCCGCCAAAGAGAACGGACTCTGTGGGCTGATTTTTCACGCTAT-3′; Mut3-R: 5′-CGATAGCGTGAAAAATCAGCCCACAGAGTCCGTTCTCTTTGGC-3′; Blue2-F:

5′-GGCCGCAAGTGGTGGCCTAACTACGGCTACACTAGAAGGACACAT 3′; Blue2-R: 5′-CGATGTGTCCTTCTAGTGTAGCCGTAGTTAGGCCACCACTTGC-3′; Repl-F:

5′-GGCCGCGGGCTGATTTTTCCATGTAT Repl-R:

5′-CGATACATGGAAAAATCAGCCCGC Rep3-F:

5′-GGCCGCGGGCTGATTTTTCCATGTGGGCTGATTTTTCCATGTGG GCTGATTTTTCCATGTAT-3′ Rep3-R:

5′-CGATACATGGAAAAATCAGCCCACATGGAAAAA

TCAGCCCACATGGAAAAATCAGCCCGC-3′ All plasmid minigenes were prepared using plasmid maxi kits from Qiagen. The identities all minigenes were confirmed by automated DNA sequencing using an ABI sequencer.

PCR Amplification of DNA Templates.

PCR from DNA templates was performed with 1-2 ng of plasmid DNA or 1-5 μg genomic DNA and Taq DNA Polymerase (Boehringer Mannheim) according to the supplier's recommendations. Amplifications were performed using a Perkin-Elmer 2400 Thermal Cycler. A typical cycle consisted of initial denaturation at 94° C. for 4 minutes, followed by 30-40 cycles of denaturation at 94° C. for 30 seconds, annealing at 65° C. for 1 minute, and extension at 72° C. for 1-2 minutes. After completion of the final cycle, a final extension was done at 72° C. for an additional 7 minutes. For amplification of templates longer than 2 kb TaqPlus (Stratagene) was used according to the manufacturers recommendations and cycles similar to those described above except that extension times were generally 5-8 minutes.

Cell Culture and Transfections.

AT3 and DT3 cells were maintained in Dulbecco's Modified Eagle Medium (DMEM, Gibco) supplemented with 10% Fetal Bovine Serum (Hyclone). All transfections were performed in 35 mm wells using 5 μl Lipofectamine (Gibco) according to the supplier's recommendations. Each well was seeded with 3×10⁵ cells 16-24 hours prior to transfection. Pilot experiments with chloramphenicol acetyltransferase (CAT) reporter plasmids demonstrated that DT3 and AT3 cells were transfected with equivalent efficiency. Transient transfections were done for 2 hours using 50 ng to 2 μg of minigene DNA, and RNA was harvested 24 hours later. Stable transfections were performed using 2 μg of minigene DNA and after 24 to 48 hours the cells were trypsinized and reseeded in 75 cm² flasks containing Geneticin (Gibco) at an active concentration of 400 μg/ml. Selection was performed until isolated colonies were obtained and no cells remained from a control transfection with a plasmid containing no neomycin resistance gene (usually 12-16 days). Pooled colonies were then harvested for RNA preparation.

RNA purification and RT-PCR analysis. Total cellular RNA was isolated from transfected cells using the method of Chomczynski and Sacchi (12). 2 μg of total RNA were heated to 100° C., chilled on ice, and reverse transcribed in a reaction volume of 20 μl. containing 50 mM Tris-HCl (pH 8.3), 75 mM KCl, 3 mM MgCl₂, 10 mM DTT, 1 mM dNTPs, 100 ng random hexamers, 2 U Rnasin (Promega), and 200 U MMLV-RT (Gibco) at 37° C. for 1 hour. Samples were then heated to 90° C. for 5 minutes, then chilled on ice. 2 μl of each reverse transcription reaction was amplified in a 100 μl PCR reaction containing 50 mM KCl, 10 mM Tris-HCl (pH 8.8), 1.5 mM MgCl₂, 0.1% gelatin, 200 μM DATP, dGTP, and TTP, 50 μM dCTP, 100 nM each primer, 10 μCi [α-³²P] sCTP , and 2.5 U Taq DNA polymerase (Boehringer Mannheim). Primers used were FGF-FB:

5′-CCCGGGTCTAGATTTATAGTGATGCCCAGCCC-3′and FGF-RB:

5′-CCCGGGGAATTCACCACCATGCAGGCGATTAA-3′ for analysis of the endogenous gene and standard T7 and SP6 promoter primers for analysis of transfected minigenes. Amplification conditions consisted of an initial denaturation at 94° C. for 4 minutes, followed by 40 cycles of denaturation at 94° C. for 30 seconds, annealing at 65° C. for 30 seconds, and extension at 72° C. for 1 minute, and a final extension at 72° C. for 7 minutes. In all amplifications reactions, a water control and a mock reverse transcription control was included which resulted in no PCR product in all experiments. PCR products were directly added to restriction endonuclease digestions with either Ava I or Hinc II (New England Biolabs). Complete digestion using this protocol was always observed. Aliquots representing equal amounts of each PCR reaction with undigested and digested PCR products were loaded directly on 5% polyacrylamide non-denaturing gels at 100V for 3-4 hours, followed by drying and exposure to Amersham Hyperfilm-MP. Phosporimager analysis was performed using a Molecular Dynamics Phosphorimager. Because equal amounts of Ava I and Hinc II digested PCR products were loaded onto each gel, quantification of exon IIIb or IIIc containing cDNAs (UBD or UCD) were obtained by using the quantification of the band at 380/377 bp which remained following Hinc II or Ava I digestion, respectively, as the numerator. The denominator consisted of the sum of the bands remaining at 380/377 bp for each digest (UBD+UCD). When these results were also expressed with the contribution of IIIb and IIIc skipped products, the average value of the 232 bp band was also used in the sum of the denominator (UBD+UCD+UD), corrected for molar equivalents. Quantification of experiments using minigenes with only one (IIIb or IIIc) internal exon was determined as the sum of the 380 or 377 bp band divided by the sum of this same band and the 232 bp band corrected for molar equivalents.

Results

Nucleotide Sequences of the Introns Flanking Alternative Exons IIIb and IIIc are Highly Conserved Between Rat and Human.

Because phylogenetic sequence comparisons often help to identify sequences with important functions, genomic DNA was sequenced from the regions of both rat and human FGF-R2 genes containing the alternative IIIb and IIIc exons, the constitutive exons located upstream and downstream of them, and the three introns between these exons. As seen in FIG. 1 the intron sizes flanking the alternative exons are roughly 1.1, 1.2 and 1.9 nucleotides in both rat and human genomic sequences. Henceforth, these exons will be referred to as intron 1, intron 2, and intron 3, respectively. The University of Wisconsin Sequence Analysis Package GAP program was used to align the rat and human sequences for direct sequence comparison. As expected, the exon sequences were highly similar and corresponded to previously reported cDNA sequences for rat and human FGF-R2 (25, 46, 65). Interestingly, the introns contained a number of regions with a very high level of sequence similarity and these regions were clustered around the IIIb and IIIc exons, whereas the intron sequences adjacent to the constitutive exons did not show an appreciable level of similarity. The rat sequence was screened and all intronic regions in which 90% of the nucleotides were identical to the corresponding human sequence for a stretch of at least 20 consecutive nucleotides were highlighted. These data are presented graphically in FIG. 1B. While some of these regions may represent evolutionary vestiges, it was also expected that regulatory sequences involved in mediating alternative splicing of these exons, which have been conserved from rat to human, would likely be represented by such conserved sequences. Thus, this information was used to direct the construction of a variety of minigenes, described below.

Minigenes pI-11-FL and pI-11-FS Recapitulate the Splicing Pattern of the Endogenous Gene in AT3 and DT3 Cells.

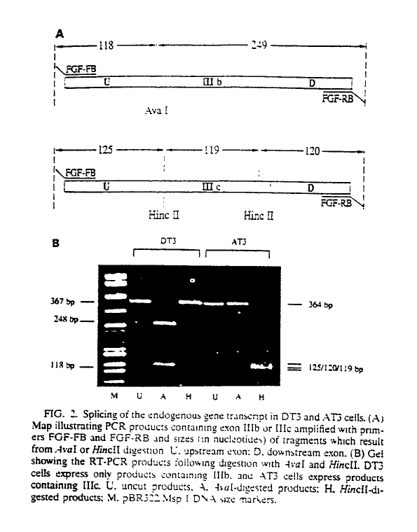

An RT-PCR based assay was used that is similar to one used by other researchers investigating splicing of human FGF-R2 to assay for the splicing pattern of exons IIIb and IIIc (14, 21). For analysis of the endogenous FGF-R2 transcript, RT-PCR was performed using primers (FGF-FB and FGF-RB) specific for the constitutive exons located upstream of IIIb and downstream of IIIc as shown in FIG. 2A. These products were separately digested with Ava I and Hinc II and analyzed by gel electrophoresis as demonstrated in FIG. 2B. Because exon IIIb contains an Ava I site not present in IIIc and exon IIIc contains two Hinc II sites not present in IIIb, IIIb inclusion is expected to result in a 367 bp product which is cut with Ava I but not Hinc II, and IIIc inclusion to result in a 364 bp product which is cut only by Hinc II. As expected, DT3 cells produce an FGF-R2 transcript which contains exclusively exon IIIb and AT3 cells consist entirely of IIIc containing transcripts; results which are consistent with the original report describing alternative splicing of FGF-R2 in these cells (63). The validity of this assay was tested by performing the same assay using titrations in which RNAs that contained only exon IIIb were mixed with RNAs containing only exon IIIc and it was observed that the proportion of each isoform seen by RT-PCR directly correlated with the fraction of the same isoform in the mixture. Thus, mRNAs containing exon IIIb or IIIc were amplified with equivalent efficiency in this assay. Furthermore, sequencing of these RT-PCR products verified that they were correctly identified by this approach.

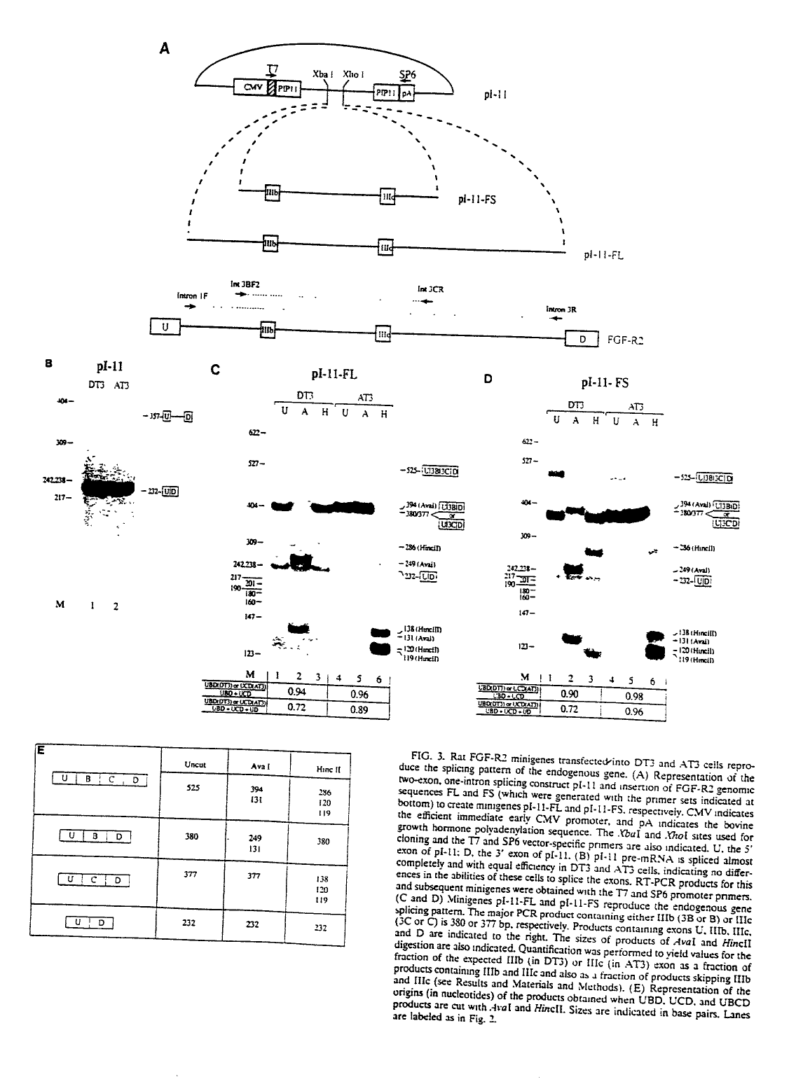

In order to further characterize the cis-elements required for maintenance of the cell type specific splicing pattern in DT3 and AT3, PCR was used to amplify genomic regions FL and FS from the rat FGF-R2 gene (FIGS. 1B and 3A). These sequences were cloned into the intron of splicing construct pI-11, which is a two exon, one intron adenovirus derived splicing construct adapted for eukaryotic expression. As seen in FIGS. 1B and 3A, the resulting minigenes, pI-11 FL and pI-11-FS, consisted of 4378 nucleotides and 1804 nucleotides of genomic FGF-R2 sequence, which represented exons IIIb and IIIc, the entire intron 2 between IIIb and IIIc and variable amounts introns 1 and 3. In the case of pI-11-FL nearly all of introns 1 and 3 are included excepting intron sequences of the normal 5′ splice site of the upstream exon/intron boundary and the polypyrimidine tract and 3′ splice site in the intron downstream of exon IIIc . In constructing pI 11-FS the sequences from introns 1 and 3 were limited to those regions closest to exons IIIb and IIIc which contained greater than 90% intron homology as described previously. In constructing these and all subsequent rat FGF-R2 minigenes, the adenoviral pPIP11 exons were substituted for the constitutive exons normally used upstream and downstream of exons IIIb and IIIc. Stable and transient transfections of these minigenes were performed in DT3 and AT3 cells using Lipofectamine and RNA was harvested 24 hours later for transient transfections or after colony formation 12-16 days later for stable transfection analysis. Labeled RT-PCR was then performed using primers (the T7 and SP6 promoter primers) specific for PCDNA3. Results from transient transfections were highly dependent on the amount of minigene DNA transfected and thus stable transfections were predominantly used due to easier reproducibility. Initial experiments using pI-11 in both DT3 and AT3 cells showed that the adenoviral strong consensus splice sites directed highly efficient splicing in both cell lines with the pre-mRNA transcript being spliced essentially to completion (FIG. 3B). In order to analyze the splicing products of pi-11-FL and pI-11-FS, the RT-PCR products were digested with Ava I and Hinc II and polyacrylamide gel electrophoresis was performed. When stable transfections with pI-11 FL and pI-11-FS were analyzed, as seen in FIGS. 3C and 3D, the primary product was 380 or 377 bp and this product contained almost exclusively exon IIIb in DT3 cells and IIIc in AT3 cells. In addition, a minor 525 bp band was also observed that corresponded to a product which contained both IIIb and IIIc as well as a 232 bp product which results when the pI-11 exons are directly spliced together. The origin of the products obtained when these products are digested with Ava I and Hinc II is shown in FIG. 3E. Digestion of the 525 bp product which contains both exons IIIb and IIIc results in a band at 394 bp, just above the location of the predominant product of 380 bp which is nearly completely digested by Ava I (see lane 2 in FIGS. 3C and 3D). Thus, this product is not to be confused with undigested 380 bp products. Although the splicing product containing both exons was seen reproducibly in all experiments, it was a minor product on a molar basis and thus for simplicity it was not included in further analysis. The amount of product seen in which both IIIb and IIIc were skipped and the pI-11 exons are directly spliced was slightly higher in DT3 cells than in AT3 cells which may in part reflect the fact that the IIIb exon contains a weaker polypyrimidine tract than that associated with exon IIIc. In order to evaluate the efficiency of usage of IIIb and IIIc in the experiments, phosphorimager analysis was used to quantify the levels of IIIb (in DT3 cells) or IIIc (in AT3 cells) containing products. Because loss of a pathway selecting either IIIb or IIIc (but not both) has two consequences, one being a switch to use of the other of these exons, or to skip both and directly splice the pI-11 exons, the results were quantified both including and excluding the contribution of this skipping pathway. These experiments using stable transfections demonstrated that the endogenous gene's pattern of regulated splicing of the IIIb and IIIc exons could be reproduced with a high degree of precision. It was also possible to demonstrate that the sequences in the exons normally located upstream of exon IIIb and downstream of IIIc, as well as most of the sequences of introns 1 and 3, are dispensable for maintenance of regulated splicing of these exons of FGF-R2.

Deletion of Nucleotide Sequences Between the Nde I and Nsi I Sites Located IN Intron 2 Causes Loss of Splicing Regulation.

To further characterize intron sequences required for splicing regulation, a series of deletions in intron 2 of pI-11-FS were constructed. The deletions tested are shown in FIG. 4A along with a map in which the regions of high rat: human homology are superimposed on the locations of the restriction enzymes used to create these deletions. The resulting minigenes were stably transfected into DT3 and AT3 cells. The results of the entire series of deletions are summarized in FIG. 4A and selected gels representing the most informative deletions are shown in FIGS. 4B and 4C. It should first be noted that a deletion from the Bcl I site at position 302 to the Nde I site at 915 which removed more than half of intron 2 did not appreciably affect appropriate splicing regulation in either DT3 cell or AT3 cells; DT3 cells used IIIb and AT3 cells used IIIc (FIGS. 4B and 4C). However, any deletion which included the sequences between the Nde I and Nsi I sites at positions 915 and 978 resulted in a dramatic loss of exon IIIb usage by DT3 cells. This loss of regulation in DT3 cells was reflected by increased usage of the IIIc exon as well as by increased skipping of both exons as seen in FIG. 4B. In addition, it was noted that a deletion from Nsi I to Stu I also caused a decrease in IIIb usage but not nearly as much as a much smaller deletion from the Nde I to Nsi I site. As demonstrated in FIG. 4C, when any of these deleted minigenes were stably transfected into AT3 cells, appropriate use of the IIIc exon was not affected, nor was any increased level of skipping observed. Because nearly identical results were obtained in AT3 cells with all manipulations of intron 2 sequences, mainly results obtained in DT3 cells are shown.

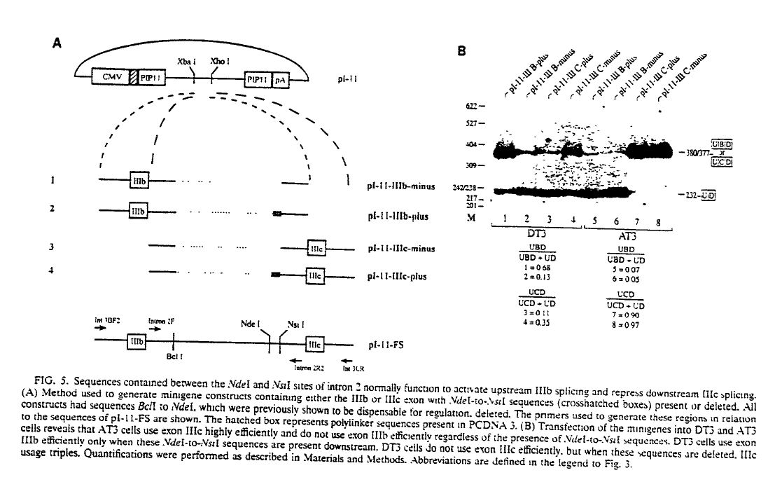

Sequences between the Nde I and Nsi I sites mediate regulation in DT3 cells by activating use of the upstream IIIb exon as well as by repressing use of the downstream IIIc exon. The results obtained with deletions in intron 2 suggested that the requirements for exon IIIc inclusion in AT3 cells are less stringent than those for IIIb inclusion in DT3 cells. In fact, with the sets of minigenes no intronic sequences outside of the conserved splice junctions or poypyrimidine tract were observed which impeded splicing of IIIc in AT3 cells. This is not surprising given the stronger polypyrimidine tract associated with the IIIc exon when compared to that of IIIb. Thus, while the possibility exists that there are other untested intron sequences or exon IIIc sequences which interact with AT3 cell-specific factors to mediate IIIc inclusion in these cells, it is expected that IIIc exon inclusion is a default splicing pathway which may only require the cooperation of factors involved in the constitutive splicing process. Thus, regulation may be achieved by proteins in DT3 cells which are able to switch the splicing pattern from exon IIIb to IIIc. Consistent with this view are the observations that several of the deletions caused not only skipping of both exons, but also a switch towards some IIIc inclusion. Therefore, if FGF-R2 mutually exclusive alternative splicing is predominantly regulated only in DT3 cells, the sequences which are involved in this regulation could be acting by activating IIIb splicing, repressing IIIc splicing, or by performing both of these functions. To investigate these alternatives, a series of minigenes were constructed in which either IIIb or IIIc (but not both) was inserted into pI-11 and the previous deletions were used in such a manner that IIIb was inserted either with (pI-11-IIIb-plus) or without (pI-11-IIIb-minus) the Nde I to Nsi I sequences located downstream, and IIIc was inserted with (pI-11IIIc -plus) or without (pI-11-IIIc-minus) these same sequences upstream (FIG. 5A). Because these minigenes only offered a choice of including an internal exon or skipping, the use of the internal IIIb or IIIc exon was quantified vs. that of the skipped product. As shown in FIG. 5B, when these minigenes were transfected into AT3 cells, the IIIc exon was included highly efficiently, and this inclusion was not affected by the presence of the Nde I to Nsi I sequences located upstream. In addition, AT3 cells did not include exon IIIb efficiently and this effect was essentially unchanged whether or not these sequences were located downstream. In DT3 cells, on the other hand, IIIb inclusion was seen to occur with fairly high efficiency, but this inclusion was largely dependent on the presence of the Nde I to Nsi I sequence located downstream; when this sequence was deleted IIIb inclusion was dramatically reduced from 68% to only 13%. In addition, it was noted that when the Nde I to Nsi I sequences were present upstream of IIIc, DT3 cells included IIIc rarely, but when these sequences were deleted, the proportion of IIIc included approximately tripled, from 11% to 35%. This data was consistent with a model in which regulation of FGF-R2 alternative splicing in DT3 cells is achieved by the interaction of a cell-specific factor or complex of factors that interact with intronic sequences in intron 2 and coordinated activation of exon IIIb splicing and repression of the stronger IIIc exon. The fact that the sequences between Nde I and Nsi I were necessary for both of these effects to occur resulted in this sequence being designated ISAR (Intronic Splicing Activator and Repressor) and indicated that this element is required for the formation of a regulatory complex which acts in DT3 cells to force the use of IIIb instead of IIIc. (The sequence between exons IIIb and IIIc is set forth in FIG. 10 (rat) and FIG. 11 (human))

A Core Nucleotide Sequence of 18 Nucleotides Within the ISAR Sequence Mediates Splicing Regulation in DT3 Cells.

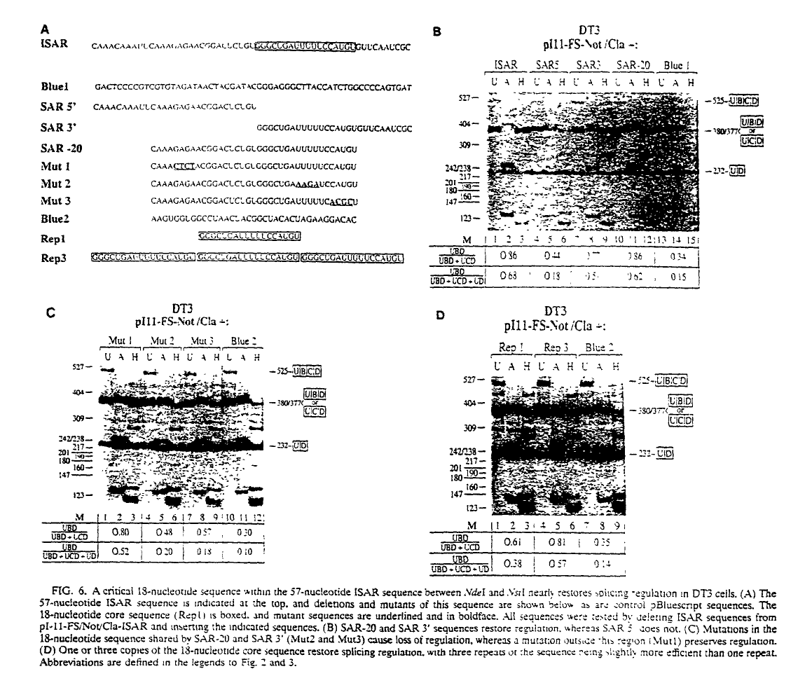

In order to further characterize the sequences in this Nde I to Nsi I ISAR element which are required for regulation, a series of deletion mutants were prepared to see which regions appeared most crucial. In order to facilitate easier manipulation of the ISAR sequence, a new minigene pI-11-FS/Not/Cla-ISAR was created in which PCR was used to engineer a Not I site directly 3′ of the Nde I site and a Cla I site was placed directly 5′ of the Nsi I site in intron 2. Thus, this minigene was identical to pI-11-FS except that the Nde I and Nsi I restriction sequences were separated from the 57 nucleotides normally located between them by the Not I and Cla I sites, respectively. The pI-11-FS/Not/Cla-ISAR was stably transfected into DT3 and AT3 cells and no differences were observed between the splicing patterns with this construct and that of pI-11 FS; splicing regulation was preserved (see FIG. 3D lanes 1-3 and FIG. 6B lanes 13). The 57 nucleotide ISAR element was then replaced with sequences consisting of parts of the ISAR sequence or containing mutations within the ISAR sequence (FIG. 6A). In addition, the ISAR element was replaced with random sequences chosen from pBluescript as controls, to verify that the effects of the deletions are a result of loss of this sequence and not due to changes in the size of the intron, and thus possibly, in the proximity of other cis-acting elements.

Minigenes were first tested in which the full ISAR sequence was replaced with only the most 5′ 29 nucleotides (SAR 5′), the 3′ 28 nucleotides (SAR 3′), or the central 37 nucleotides (SAR 20), as outlined in FIG. 6A. Also a 57 nucleotide sequence from pBluescript (Bluel) was introduced as a control for the size of ISAR. When these constructs were stably transfected into AT3 cells, highly efficient, nearly exclusive use of exon IIIc was observed. In DT3 cells, a dramatic reduction in IIIb inclusion was observed with the control Bluel sequences as well as with SAR 5′ (FIG. 6B). SAR 3′ and SAR-20, however, nearly restorated splicing regulation, although IIIb inclusion with SAR 3′ was slightly less efficient than with SAR-20. Because SAR 20 restored splicing regulation almost to levels seen with full length ISAR, several mutations (Mut 1, Mut2, and Mut3) within this sequence were tested in which 4 consecutive nucleotides were mutated within SAR-20 by replacing purines with pyrimidines or vice versa. In addition, because SAR-20 and SAR 3′ both nearly restored regulation, the effect of the 18 nucleotides these sequences shared (Repl, boxed in top sequence, FIG. 6A) was tested as well as three repeats of this sequence (Rep3). A second negative control was also included from a different part of pBluescript (Blue 2) which was 37 nucleotides in order to correspond to the size of SAR-20. A loss of regulation in DT3 cells was noted when either of the Bluescript sequences were used as well as both mutations, Mut2 and Mut 3, which were located within the 18 nucleotides which appeared to be most critical (FIG. 6C). However, Mut1, Rep1, and Rep 3 sequences all were capable of at least partially restoring splicing regulation (FIGS. 6C and 6D). In fact, three repeats of this 18 nucleotide region appeared to be slightly more efficient than one repeat, although not better than full length ISAR (FIG. 6D). Thus, this 18 nucleotide region contained most of the sequences required for regulation by the ISAR element, although given a slightly decreased level of restoration of regulation by SAR 3′ and Repl compared to ISAR or SAR-20, there may be a minor contributing role of other sequences within ISAR.

Regulated Splicing in DT3 Cells is Dependent on a Titratable Factor or Factors.

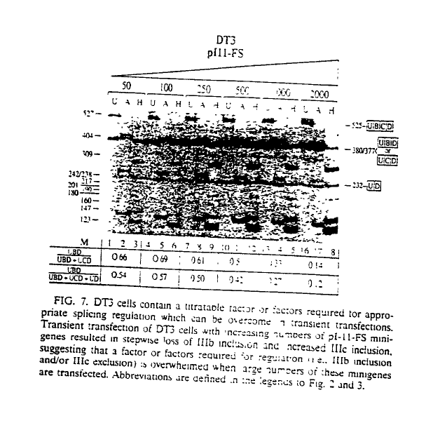

In the initial experiments using FGF-R2 minigenes pI-11-FS and pI-11-FL in transient transfections using 2 μg of plasmid DNA, it was noted that AT3 cells utilized exclusively the IIIc exon as was observed in stable transfections; however, the DT3 cells used the IIIc exon predominantly as well, with only a minor component of IIIb usage. Because transfection using lipofectamine is highly efficient, it was hypothesized that splicing regulation was not being seen in these experiments because the amount of RNA produced in these transient transfections may have been overwhelming the regulatory factors present in DT3 cells. Therefore, using pI-11-FS, the amount of plasmid DNA used in these transfections of DT3 cells was titrated as shown in FIG. 7. It can be seen that when amounts of DNA in the range of 50 to 100 ng were tested, it was impossible to achieve nearly (but not quite) the same degree of IIIb inclusion as that obtained with stable transfections and the endogenous gene. As the amount of DNA used in the transfection is increased a stepwise reduction in IIIb inclusion is seen. In fact a similar effect was previously observed using human FGF-R2 minigenes, and in this case, curiously, the effect was abrogated through maintenance of an open reading frame in the minigene (Gilbert et al, Mol. Cell. Biol. 13:5461-5468 (1993)). Transient transfection of a minigene which does not contain ISAR (pI-11-FS ΔNde I/Nsi I) resulted in nearly exclusive use of the IIIc exon even using only 50 ng of DNA. Thus, ISAR was likewise required for maintenance of splicing regulation in transient transfections. However, this regulation is compromised when larger amounts of minigene are transfected, most likely due to overtitration of a factor or factors required for IIIb inclusion.

The Core Sequences of ISAR are Highly Homologous with a Similar Sequence in Human FGF-R2 Shown to Mediate IIIb Activation in Human Cells Which Express FGF-R2 (IIIb).

Recently, Del Gatto and colleagues (Del Gatto et al, Mol. Cell. Biol. 17:5106-5116 (1997) described several sequences located in the same region of the human gene between exons IIIb and IIIc which were required for upstream IIIb activation, although effects of these sequences on IIIc repression were not characterized. In addition several other intronic sequences have been shown to also be required for proper splicing regulation as well as exon sequences in IIIb (Del Gatto et al, Mol. Cell Biol. 15:4825-4834 (1995), Del Gatto et al, Nucleic Acids Res. 24:2017-2021 (1996)). These investigators proposed that a sequence element similar to the 18 nucleotide core sequence in the rat ISAR sequence could form an RNA secondary structure with another important intronic sequence, IAS2, approximately 800 nucleotides upstream from this sequence. It was further proposed that this secondary structure was a necessary element involved in exon IIIb activation. Interestingly, as shown in FIG. 8, the rat sequences corresponding to both of these elements are highly similar to the human sequence. It is interesting to note that in all of the rat minigenes tested here, including those with only one of the two exons, the corresponding upstream sequence was always included. Thus, possible synergistic effects of these separate sequences on FGF-R2 splicing regulation, whether or not they function via formation of a secondary structure, may be involved in the repression of IIIc as well as upon IIIb activation.

### EXAMPLE 2

### Preparation of FGF-R2 Isoform Specific Antibodies

In order to generate antibodies specific for the FGF-R2 IIIb and IIIc isoforms, glutathione-S-transferase (GST) fusion peptides were produced. The FGF-R2 isoforms are generated by alternative splicing of the FGF-R2 IIIb and FGF-R2 IIIc exons, both of which encode nearly 50 amino acids and comprise the second half of the third extracellular immunoglobulin-like domain. Therefore, the polymerase chain reaction was used to amplify the sequences comprising exons FGF-R2 IIIb and FGF-R2 IIIc. The resultant DNA products were then cloned into the Eco RI and Bam HI sites of pGEX-TK (Pharmacia). The cloning was designed such that the sequences from these exons were aligned in frame such that when the plasmid was expressed in bacterial cells, the products would consist of GST fusion peptides expressing the amino acids normally encoded by these alternative exons. The identity of these cloned products was confirmed by DNA sequencing. These plasmids were transformed into protease deficient BL21 E. coli and expression induced by IPTG following a standardized protocol. After purification of the expressed GST fusion peptide by glutathione agarose affinity chromatography, the purified peptides were analyzed by SDS-PAGE electrophoresis and silver staining which confirmed that pure peptides were isolated. These two purified GST peptides, GST-IIIb and GST IIIc, were then delivered to Research Genetics (Huntsville, Ala.) for injection into rabbits and production of polyclonal antisera (P.O. Number PC 171555). High titer rabbit antisera against the respective GST-IIIb and GST-IIIc peptides were obtained.

As a first step towards determination of the specificity of these antibodies for their respective peptide targets, the ability of these antisera to recognize the limited peptide sequences which were used to generate antibodies was examined. Because the antisera are expected to also contain non-specific antibodies against the GST moiety, the same FGF-R2 IIIb and FGF-R2 IIIc peptide encoding sequences were PCR amplified and cloned into pMAL-C2 expression system (New England Biolabs). The resultant Maltose Binding Protein (MBP) fusion peptides were then expressed in E. coli hosts and purified over an amylose column. These fusion peptides with FGF-R2 IIIb and FGF-R2 IIIc encoded peptides were then analyzed by SDS-PAGE and immunoblotting using anti GST-IIIb and anti-GST-IIIc antisera. Prior to Western immunoblot analysis, IgG antibodies were partially purified from the antisera and pre-immune sera using protein A beads. The results of this experiment demonstrated that the antibodies were, in fact able to specifically recognize the FGF-R2 derived peptide sequences. Both pre-immune antisera from rabbits did not recognize either of these peptides. Anti-GST-IIIb antibodies specifically recognized the MBP-IIIb, but not the MBP-IIIc fusion peptide. Conversely, anti-GST-IIIc antibodies were specific for MBP-IIIc, but not MBP-IIIb.

All documents cited above are hereby incorporated in their entirety by reference.

One skilled in the art will appreciate from a reading of this disclosure that various changes in form and detail can be made without departing from the true scope of the invention.

What is claimed is:

1. A method of determining the metastatic potential of a prostate tumor in a patient comprising assaying said tumor for the expression of the IIIc isoform of FGF-R2, wherein predominant expression of the FGF-R2 IIIc isoform relative to the FGF-R2 IIIb isoform indicates an increase in metastatic potential of said tumor.

2. The method according to claim 1 wherein said method comprises assaying for the presence of an FGF-R2 IIIc transcript.

3. The method according to claim 1 wherein said method comprises assaying for the presence of the FGF-R2 IIIc isoform.

4. The method according to claim 3 wherein said method comprises contacting a sample of said tumor with an antibody specific for the FGF-R2 IIIc isoform and determining the amount of a complex formed between said antibody and said FGF-R2 IIIc isoform.
